(19) United States
(12) Patent Application Publication
(10) Pub. No.: US 2008/0268097 A1
(43) Pub. Date: Oct. 30, 2008

(54) COCOA INGREDIENTS HAVING ENHANCED LEVELS OF STILBENE COMPOUNDS AND METHODS OF PRODUCING THEM

(76) Inventors: W. Jeffrey HURST, Mt. Gretna, PA (US); Kenneth B. MILLER, Palmyra, PA (US); David A. STUART, Mt. Gretna, PA (US)

Correspondence Address: WILEY REIN LLP, 1776 K. STREET N.W., WASHINGTON, DC, 20006, US

(21) Appl. No.: 12/110,943

(22) Filed: Apr. 28, 2008

Related U.S. Application Data
(60) Provisional application No. 60/924,012, filed on Apr. 26, 2007.

(51) Int. Cl. A23G 1/42; C12N 5/06; C12N 9/99; C07C 39/21; A23G 1/30; A23G 1/32

(52) U.S. Cl. 426/45; 435/375; 435/184; 568/729; 426/546; 426/248

(57) ABSTRACT

The invention provides methods of making food products and ingredients having enhanced levels of natural stilbene compounds, such as resveratrol. Chocolate liquor, cocoa powders, and cocoa extracts having enhanced levels of resveratrol prepared from cacao beans are disclosed. The invention also provides methods of treatment using compositions and ingredients having enhanced natural stilbene content.

### FIELD OF THE INVENTION

The invention relates to food products and ingredients that contain enhanced levels of stilbene compounds and methods of making and using them. More specifically, the invention relates to cocoa ingredients having enhanced levels of stilbene compounds, such as resveratrol, methods of making cocoa ingredients having enhanced levels of stilbene compounds, and food products containing such ingredients.

### RELEVANCE OF THE INVENTION AND DESCRIPTION OF RELATED ART

Stilbene compounds have a common structure of two benzene molecules linked by a two carbon ethylene bridge (Table 1). Hydroxylated derivatives of stilbenes are known as stilbenoids. A common stilbenoid is resveratrol, a compound of the phytoallexin family that is synthesized by certain plants (Table 2). Bioactive resveratrol is known by the chemical name trans-resveratrol, cis-resveratrol, 3,4′,5-stilbenetriol, trans-3,4′,5-trihydroxystilbene, or [2-[4-hydroxyphenyl] ethenyl]benzene-1,3 diol.

TABLE 1
Structure of Stilbene

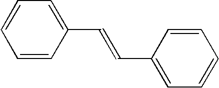

TABLE 2
Structure of Resveratrol

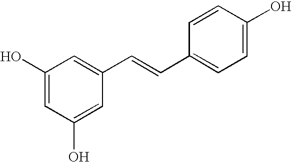

When glucosylated in the 3-position, resveratrol becomes trans-piceid (Table 3). Both compounds are absorbed by the body and have physiological importance.

TABLE 3
Structure of Trans-Piceid

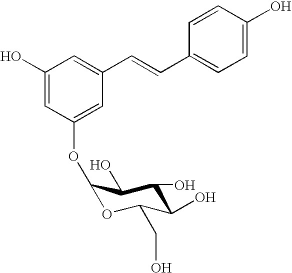

Other related stilbenes include pterostilbene (Table 4) and piceatannol (Table 5).

TABLE 4
Structure of Pterostilbene

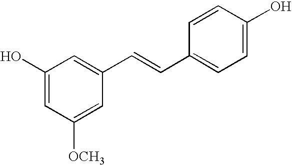

TABLE 5
Structure of Piceatannol

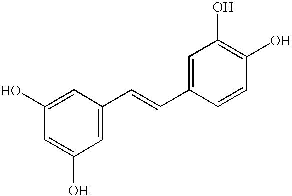

The most important sources of resveratrol in the human diet are red wine, peanuts, tomatoes, berries, and Itadori, a Japanese herbal tea. The potential importance of resveratrol in the diet was first examined in association with the so-called “French Paradox” studies that show that the typical French diet, high in fats but also high in red wine consumption, results in a lower incidence of cardiovascular disease (CVD) compared to the American diet. A hypothesis for the lower risk associated with the French diet was that compounds in red wine contribute to the lower risk of CVD. Later, chemists identified resveratrol in relatively high concentrations in red wine, especially in wines produced from pinot noir grapes. Several surveys showed that red wine is highest in resveratrol compounds, followed by red grape juice, peanuts and tomato skins. Other studies have implicated resveratrol as an anti-aging compound, and as an anti-cancer agent, among other effects.

The compound pterostilbene is present in berries, especially those of the Vaccinium genus, such as blueberries and cranberries. Pterostilbene is also found in grapes. Pterostilbene has been shown to have antioxidant, anti-cancer, anti-diabetic, and cholesterol-lowering properties.

A report by Counet et al. (Food Chem. 98: 649-57 (2006)) identified resveratrol and piceid in a single sample of cocoa liquor and dark chocolate. However, the levels of resveratrol reported are quite low, especially in comparison to wine.

Thus, methods of processing and identifying enhanced levels of natural stilbene compounds, such as resveratrol, in cocoa products that can be beneficially used in supplements, food, and beverages have not been described.

### BRIEF SUMMARY OF THE INVENTION

The invention, in one aspect, satisfies a need for beneficial stilbene compound levels in widely available consumable products, and especially products or beverages without alcohol. In combination with other known compounds found in cocoa, the new cocoa products, chocolates, and food ingredients and products advantageously provided by the invention create new possibilities for producing or supplementing foods with beneficial levels of natural stilbene compounds.

The invention relates to methods of treating, selecting, and/or processing cacao seeds, commonly referred to as beans, to make cocoa ingredients, extracts, or products having improved properties or characteristics. In particular, the invention relates to methods of producing cocoa products, extracts or ingredients having enhanced levels of natural stilbene compounds, and, in particular, resveratrol and piceid. The invention also relates to novel cocoa-containing food, supplements, and beverages having enhanced levels of natural stilbene compounds.

It is an object of the invention to provide methods of selecting and/or processing cocoa beans for producing cocoa ingredients having enhanced levels of beneficial natural compounds. In one example, stilbene compounds or stilbene oligomer compound levels, and, in particular, resveratrol and piceid levels.

It is a further object of the invention to provide cocoa ingredients, including chocolate liquor, cocoa powder, low fat cocoa powder, defatted cocoa powder, and cocoa extracts having enhanced levels of resveratrol and/or piceid, and food products containing or made from the cocoa ingredients.

Cocoa ingredients having enhanced levels of resveratrol are ingredients derived from unfermented cacao beans, or from samples of cacao beans having more than 10% unfermented beans. Cocoa ingredients having enhanced levels of resveratrol also include ingredients derived from cocoa beans that have been treated to intentionally cause a biochemical stress in the beans or to enhance stilbene levels, such as with UV light or by treatment with any of the other methods or combinations of methods described herein.

In particular embodiments, the invention comprises methods of enhancing the level of trans-resveratrol and/or trans-piceid present in a food product, where a sample of cacao beans is selected having about 1 μg/g or more of trans-resveratrol and/or 3.5 μg/g or more of trans-piceid, and/or 5 μg/g or more of total resveratrol, and supplementing a food product with non fat cocoa solids produced from these beans. In one aspect, the cacao beans used to produce non fat cocoa solids comprise more than 10% unfermented cacao beans. In other aspects, the cacao beans used comprise more than 10% slaty cacao beans or a mixture having more than 10% slaty and purple cacao beans. The selected beans can be made into a number of cocoa compositions, such as cocoa liquor, cocoa powder, low fat cocoa powder, defatted cocoa powder, and a cocoa extract. The beans can also be roasted and treated with alkali, or “Dutched,” as known in the art. There are numerous food or beverage products one could make from the cocoa compositions of the invention, including but not limited to a chocolate product, a milk chocolate product, a dark chocolate product, a semisweet or bittersweet chocolate product, a chocolate-flavored product, a chocolate confectionery, a chocolate-flavored confectionery, a beverage, a chocolate beverage, a chocolate-flavored beverage, a dietary supplement, a chocolate-coated product, a low fat chocolate product, a baked chocolate product, such as a cake, brownie, or bread product, or a low-sugar chocolate product.

Specific cocoa compositions also included in the invention are chocolate liquors having about 0.4 μg/g to about 0.7 μg/g or more of trans-resveratrol, about 4.2 μg/g or more of piceatannol, about 5.6 μg/g to about 6 μg/g or more of trans-piceid and/or about 6.4 μg/g or more of total resveratrol. The invention also includes cocoa powders having about 0.7 μg/g to about 1.3 μg/g or more of trans-resveratrol, about 6.6 μg/g to about 10.6 μg/g or more of trans-piceid, about 4.2 μg/g or more of piceatannol, and/or about 7 μg/g to about 12 μg/g or more of total resveratrol. The cocoa compositions can be treated with alkaline conditions as known in the art.

The invention also includes cocoa compositions that are an extract, or a food ingredient extract, made from the treated or selected beans described herein. In one aspect, the extracts of the present invention may be added to a food product or beverage without affecting the flavor, mouthfeel, or other physical or taste attributes of the food product or beverage. In another aspect, the methods of the invention may be used to make a cocoa extract from which inert and other undesirable compounds have been removed, such as starch, fiber, or theobromine.

In another aspect of the invention the level of stilbene compounds, such as resveratrol, is increased during processing of the cocoa into its ingredients by selecting methods that preserve the most total stilbene compounds in the ingredients. In the case of roasting Mexican cacao lavado beans at a medium roast, about 25% of the total resveratrol is lost as a result of roasting. Methods that preserve higher levels of stilbene compounds could include low temperature vacuum drying up to about 85° C. product temperature, and/or optimizing the conditions for roasting of beans including choosing appropriate time, temperature and terminal moisture levels for the processed beans to result in increased levels of stilbenes. The roasting temperature is preferably no greater than 100° C. product temperature. Other methods could include steam, steam pasteurization, dry heating, dry pasteurization, acid treatments, such as acid bath treatment at pH 1 to about pH 4, chemical or physical (pressure or irradiation) treatment of the nibs or beans rendering them microbiologically safe for consumption. In so doing, the goal is to improve the yield of stilbene compounds and/or beneficial stilbene compounds (i.e., those possessing one or more beneficial health effects as noted herein or known in the art), in particular resveratrol and/or piceid, in the processed ingredients. Thus, the invention specifically includes methods of processing cacao bean samples, especially those containing Mexican cacao lavado beans and/or any other unfermented cacao bean source, such as unfermented beans originating from Guatemala or Venezuela, in order to increase or optimize the levels of stilbene compounds, and especially resveratrol, in a resulting cocoa product compared to conventional processing methods and conventional bean samples, where the conventional methods are as described in B. Minifie, Chocolate, Cocoa, and Confectionery, 3d Ed., Aspen Publishers. In particular, the invention includes methods of increasing or optimizing the levels of any naturally occurring stilbene compounds in cacao beans or products made from beans, including compounds having beneficial health effects, such as, for example, trans- and cis-resveratrol, trans- and cis-piceid, trans- and cis-pterostilbene, and trans- and cis-piceatannol, other natural stilbene compounds and oligomers of stilbenes such as viniferin, pallidol, and astilbin, and methods of enhancing the levels of such compounds compared to conventional processing methods.

The invention also includes methods of enhancing the level of stilbene compounds in a cocoa composition, comprising preparing a cacao bean sample having more than 10% washed, unfermented cacao beans, and processing the bean sample into a cocoa composition to produce a non fat cocoa solids-containing ingredient. More specific methods include selecting unfermented cacao beans, irradiating the beans with UV light, and preparing a cocoa composition from the beans to enhance levels. The UV treatments typically are before, during, immediately after, and/or shortly after harvesting the pods. However, UV treatments can occur at multiple steps in the process. After washing, the beans can be easily treated with UV during a drying step or a separate UV treatment step. Preferably, UV-B light or UV-C light is used, and lamps emitting UV at about 254 nm wavelength are available for these purposes. The UV light treatment can occur for about one minute, or between about 1 minute to about 15 minutes.

A further or additional method of enhancing the levels of stilbene compounds, especially resveratrol and/or piceid, in products made from cacao beans comprises selecting unfermented cacao beans and treating them with one or more of: inoculating with a fungus or microbe; inoculating with Botrytis; storage for 1 or more days at about ° C.; using a percentage of moldy beans; treatment with ozone; treatment with aluminum chloride; and treatment with aluminum sulfate. These treatment methods can be done individually, or in a certain succession of any combination of them, and can each or any combination be combined with a treatment with UV light.

A further aspect of the invention is to stimulate the production of stilbene compounds, in particular resveratrol and/or piceid, by taking advantage of the plant's natural ability to turn on its own disease response mechanisms by pre-exposing the cacao plant and/or its pods to inactivated pathogens or other microbes, such as non-pathogenic organisms, to trigger the plant defense systems that are known to be associated with resveratrol synthesis. For example, cocoa plants could be exposed to inactivated pathogenic bacteria or fungi, or non-pathogenic Tricoderma sp. or Bacillus sp. from about 20 to about 60 days in advance of harvesting pods to stimulate the plant disease response system and increase the level of resveratrol in the plant and pods. In a similar manner, cocoa plants could be treated with chemicals that are involved in the plant disease response system to trigger the plant's disease response system without inoculating the plant with virulent disease-causing organisms. For example, cocoa plants could be treated with salicylic acid or derivatives that are commercially available, jasmonic acid or its derivatives, or ethylene or ethreal (a commercially available spray that releases ethylene inside the plant), or other plant elicitor compounds involved in resistance to pathogens or stress. Thus, methods of the invention include treating plants to trigger the plant's own disease response systems and causing the plant to synthesize added amounts of stilbene compounds, such as resveratrol, without inoculating the plant with virulent disease-causing organisms.

In another aspect, the invention includes methods of stimulating nitric oxide generation, methods of inhibiting the enzymatic activity of COX-1 or COX-2, and methods of decreasing NADH/NADPH oxidase activity in cells, animals, human cells and tissue by administering an ingredient or composition having enhanced levels of stilbene compounds, such as resveratrol, the ingredient prepared by selecting a sample of cocoa beans having more than 10% unfermented beans and processing the bean sample into a cocoa composition having non fat cocoa solids in an edible form. The compositions of the invention can be prepared by selecting a sample of cocoa beans having more than 10% unfermented beans, reducing the beans to a powder, defatting the powder, and extracting natural stilbene compounds from the powder. The invention further includes pharmaceutical compositions containing natural stilbene compounds prepared from cacao beans. A variety of pharmaceutical compositions can be prepared by methods known or apparent to those skilled in the art and are described in more detail in, for example, Remington's Pharmaceutical Science, 17th ed., Mack Publishing Company, Easton, Pa. (1985), and the 18^{th} and 19^{th} editions thereof, which are incorporated herein by reference,

Throughout this disclosure, applicants refer to journal articles, patent documents, published references, web pages, and other sources of information. One skilled in the art can use the entire contents of any of the cited sources of information to make and use aspects of this invention. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. The description and examples that follow are merely exemplary of the scope of this invention and content of this disclosure and do not limit the scope of the invention. In fact, one skilled in the art can devise and construct numerous modifications to the examples listed below without departing from the scope of this invention.

### DETAILED DESCRIPTION OF EXEMPLARY EMBODIMENTS

In one aspect the invention involves the use of cacao beans from any source, and products made from them or derived from them. The terms “cocoa extract,” “cocoa bean composition,” and “cacao bean composition” can be any of a variety of products and combinations of the cacao bean-derived products noted in this disclosure. “Cocoa bean composition,” “cacao bean composition” and “cocoa products” are essentially interchangeable and mean a product made from a cacao bean. A “cacao bean sample” or a “cocoa bean sample” is a collection of cacao beans or the nibs of such beans from a desired set of sources or set of processing conditions. In addition, combinations of cocoa products or cocoa extracts involving cacao beans treated, processed, or selected under conventional methods can be combined with cacao bean compositions of the invention. These compositions and extracts can be used in any cocoa ingredient, which in turn can be used in any composition or product for human consumption, including foods, confections, beverages, and supplements.

Cocoa powder, as understood in the art, contains approximately 10-25% lipid fraction (cocoa butter). However, all or a percentage of the fat can be removed from the powders by pressing, by solvent or supercritical solvent extraction or any number of other methods, as known in the art. Thus, natural, defatted and/or low fat or non-fat cocoa powders are specifically included in the cocoa products or ingredients of the invention. Furthermore, the preferred cocoa powders, cocoa bean compositions, and cocoa products of the invention can be treated with alkali, a process also known as “Dutching,” in a manner such that the level of total resveratrol, trans-resveratrol or trans-piceid will be greater than the levels in fermented cocoas. Other cocoa products, such as breakfast cocoa, cocoa extracts, and chocolate liquor can also be produced from the invention.

As one of skill in the art understands, a certain amount or percentage of cocoa solids in a food ingredient can be achieved, inter alia, by using or adding an amount of cocoa powder, chocolate liquor, or other chocolate or cocoa ingredient containing the requisite amount of cocoa solids. Similarly, a certain amount or percentage of natural cocoa in a food ingredient can be achieved, inter alia, by using or adding an amount of cocoa powder, cocoa extract, chocolate liquor, or other chocolate or cocoa ingredient. Other nutritional, therapeutic, or preventative ingredients can be added as well, as known in the art.

The cocoa compositions and products of the present invention contain enhanced levels of total resveratrol, and the compound trans-resveratrol and/or its glucoside trans-piceid. As used herein, the term “resveratrol” refers collectively to trans-resveratrol or cis-resveratrol or a combination of both. The term “piceid” refers collectively to trans-piceid or cis-piceid or a combination of both. The term “total resveratrol” refers to the total amount of trans-resveratrol and trans-piceid present or detectable. The terms “stilbene compound” and “natural stilbene compound” refer collectively to trans- and cis-resveratrol, trans- and cis-piceid, trans- and cis-pterostilbene, trans- and cis-piceatannol and any other stilbene compounds and stilbene oligomers found naturally in cacao, including those having a beneficial health effect in humans. As discussed here, one can detect an increase in the levels of one or more stilbene compounds in unfermented cacao beans compared to fermented cacao beans.

As noted above, recent studies have reported potential benefits of dietary resveratrol with respect to metabolic syndrome and food consumption. Resveratrol, (3,4′,5-trihydroxystilbene), is a member of the stilbene family and has been associated with the French Paradox (Renaud, S.; Gueguen, R. The French Paradox and wine drinking; Novartis Foundation Symposium, 1988, 16: 208-174). Studies also have shown that consumption of resveratrol increases vasodilation (Orallo, F.; Álvarez, E.; Camiña, M.; Leiro, J M.; Gómez, E.; Fernández, P., The possible implication of trans-resveratrol in the cardioprotective effects of long term moderate wine consumption, Mol. Pharmacology. 2002, 61: 294-302), inhibits platelet aggregation and coagulation (Bertelli, A A.; Giovanni L.; Gianessi D.; Migliori, M.; Bernini, W.; Fregoni, M.; Bertelli, A., Anti-platelet activity of synthetic and natural resveratrol in red wine, Int. J. Tissue React. 1995, 17:1-3) as well as modifies eicosanoid synthesis and lipoprotein metabolism (Pace-Asciak, CR.; Hahn, S.; Diamandis, EP.; Soleas, G.; Goldberg, D M, The red wine phenolics trans resveratrol and quercetin block platelet aggregation and eicosanoid synthesis: Implications for protection against coronary heart disease, Clin Chim Acta 1995, 235: 207-219). Furthermore, some hypothesize that resveratrol's structure suggests phytoestrogenic potential which may further contribute to its potential cardioprotective effects (Gelm, B.; McAndrews, J.; Chien, P.; Jameson, J., Resveratrol, a polyphenolic compound found in grapes and red wine is an agonist for the estrogen receptor, Proc. Nat. Acad,. Sci USA, 1997, 94: 14138-43). These studies have not addressed cocoa products as a beneficial source of high levels of stilbene compounds in the diet. As shown below, the total resveratrol present in conventional cocoa powder or chocolate products averages between 0.09 μg/g to 1.85 μg/g. The “average” and “range” of the samples used for Table 6 are the average total resveratrol present, and the ranges are representative of the top selling United States brands in the six categories of products listed.

TABLE 6
Total resveratrol content of selected chocolate
and cocoa-containing products

Total Resveratrol
(μg/g)

| Product Type | Average | Range |
|---|---|---|
| Cocoa Powder | 1.85 | 1.20-2.30 |
| Baking Chocolate | 1.24 | 0.87-1.47 |
| Semisweet Baking Chips | 0.54 | 0.33-0.66 |
| Dark Chocolate | 0.35 | 0.25-0.52 |
| Milk Chocolate | 0.10 | 0.05-0.17 |
| Chocolate Syrup | 0.09 | 0.06-0.11 |

From these conventional products, cocoa powder has the highest levels of total resveratrol, followed by baking chocolate, semi-sweet baking chips, dark chocolates, milk chocolates, and chocolate syrup. The level of total resveratrol is proportional to the level of non fat cocoa solids in each product category. Even in these conventional preparations, the level of total resveratrol in cocoa powder, baking chocolate, and dark chocolate, when expressed as standard serving sizes, are second only to red wine as a source of resveratrol, as shown in Table 7.

TABLE 7
Total resveratrol contents per serving of wine,
peanut, and cocoa-based products

| | | | TotalResveratrol/Serving | |
|---|---|---|---|---|
| | Serving | | (μg) | |
| Product | Size | | Average (Range) | |
| Red Wines | 150 | ml | 656 | (225-1170) |
| Red Wines (Calif.) | 150 | ml | 263 | (34-872) |
| Roasted Peanuts | 28 | g | 1.5 | (0.5-2.2) |
| Peanut Butter | 32 | g | 9.6 | (4.4-15.1) |
| Cocoa Powder | 10 | g | 18.5 | (12.0-22.7) |
| Baking Chocolate | 15 | g | 18.5 | (9.9-22.1) |
| Dark Chocolate | 40 | g | 14.1 | (10.0-17.2) |
| Semisweet Baking | 15 | g | 7.9 | (4.9-9.0) |
| Chips | | | | |
| Milk Chocolate | 40 | g | 4.1 | (2.0-6.8) |
| Chocolate Syrup | 39 | g | 3.4 | (2.3-4.3) |

Compared to red wine in particular, these products contain relatively low levels of total resveratrol. The products and compositions of the present invention contain enhanced levels of trans-resveratrol, trans-piceid and/or total resveratrol compared to these conventional cocoa products. In particular, cocoa compositions, products or ingredients made from unfermented, freshly dried cocoa beans according to the invention have the highest levels of resveratrol and piceid, as shown by a comparison to light, medium and heavily fermented beans (see Table 8; “Fermentation Index” here and elsewhere refers to the method in Shamsuddin et el, in “Cacao Biotechnology Symposium,” pages 55-74, 1986, Ed. Dimick, Dept of Food Science College of Agriculture The Penn State Univ.). Specifically, unfermented cocoa beans have 8.46 μg/g total resveratrol, while lightly fermented beans have 4.12 μg/g total resveratrol, medium fermented beans have 3.65 μg/g total resveratrol and heavily fermented beans have 3.59 μg/g and 3.66 μg/g total resveratrol.

TABLE 8
Total resveratrol levels in freshly dried and fermented cocoa beans.

| | | trans- | | Total |
|---|---|---|---|---|
| | Fermentation | resveratrol | trans-piceid | Resveratrol |
| Bean Origin | Index | (μg/g) | (μg/g) | (μg/g) |
| Mexico Cacao | 0.38 (None) | 2.18 | 6.28 | 8.46 |
| Lavado | | | | |
| Sulawesi | 0.79 (Light) | 0.70 | 3.24 | 4.12 |
| Ivory Coast | 0.97 (Medium) | 0.41 | 3.24 | 3.65 |
| Papua N. | 1.38 (Heavy) | 0.39 | 3.27 | 3.66 |
| Guinea | | | | |
| Jamaica | 1.44 (Heavy) | 0.44 | 3.15 | 3.59 |

Accordingly, in one aspect, the invention involves the novel use of unfermented cacao beans and/or collections or samples of cacao beans having certain desirably enhanced levels of resveratrol and/or piceid. In particular, the cacao beans or samples of them can have about 5 μg/g or more of total resveratrol, and these beans can optionally be roasted or processed to produce a cocoa composition of the invention, such as a cocoa ingredient for a food, beverage, or supplement product. In general, the samples of cacao beans will comprise a higher level of unfermented beans than is conventionally used for cocoa processing, and samples with about 10% unfermented beans or more can lead to detectably higher trans-resveratrol or total resveratrol levels, for example.

Considering that plants can synthesize resveratrol in response to environmental stresses, such as fungal or bacterial infections, and that the fermentation process exposes cocoa beans to the stresses of high populations of microorganisms, high heat, and alcohol (See King, et al., Bioactivity of Resveratrol, Comp. Rev. Food Sci. & Food Safety, 5: 65-70 (2006)), these results are surprising and unexpected.

The comparative stresses endured during the fermentation process are relatively high compared to the unfermented bean. For example, mature pods from cacao trees are typically harvested with about thirty to fifty cacao beans per pod surrounded by a mucilaginous pulp. While in the pod, the beans are in a sterile environment, free of fungi and bacteria. After harvesting, the beans and adhering pulp are removed from the pods and are either heaped onto and covered with banana leaves or other leaves or transferred to fermentation boxes. In the early stages of fermentation, organisms such as yeast and fungi inoculate the sweet pulp surrounding the beans. Sugars from the pulp are broken down to form small amounts of ethyl alcohol and lactic acid, and the temperature rises to about 113° F. As the fermentation proceeds, the cocoa beans are exposed to high populations of microorganisms, high heat (up to 140° F.), alcohol and drying. As the alcohol levels increase, the microbial population changes from yeast to bacteria. The bacteria continue to multiply and convert the alcohol to acetic and lactic acids. Eventually, the combination of stresses from microbes, alcohol, heat and acid kills the beans rendering them incapable of germination. Considering these factors and stress conditions, one would generally conclude that resveratrol levels would be higher in cocoa beans undergoing some level of fermentation compared to unfermented beans. Contrary to this assumption, we have found that the unfermented cocoa beans contain the highest levels of total resveratrol.

In one aspect, the present invention comprises methods of making cocoa ingredients having enhanced levels of natural stilbene compounds, such as resveratrol and piceid, by exposing cacao plants and/or cocoa beans to one or more treatments or environmental stresses before, during or shortly after harvesting. Exposure of an environmental stress before, during or shortly after harvesting can increase the levels of resveratrol in cocoa beans. One example of an environmental stress that may increase resveratrol or piceid levels in cocoa beans is exposure to a fungal infection, such as, for example, the noble rot infection of the fungus Botrytis cinerea. Other treatments or environmental stresses that may be used within the scope of the present invention to increase resveratrol and/or piceid levels in cacao plants include exposure to a microbial infection, high temperatures, and ultraviolet radiation. Furthermore, collections or samples of cacao beans that have been treated or exposed to environmental stresses according to the invention can be combined with other beans to produce a cocoa composition with enhanced levels of resveratrol. Collections or samples of cacao beans with 10% or more treated unfermented beans of the invention can enhance levels to a beneficial amount. Thus, combinations of beans or samples of beans that have a total resveratrol level of about 3 μg/g, or about 5 μg/g, or about 6 μg/g, or about 7 μg/g, or about 8 μg/g or more are specifically included in the invention, as well as cocoa compositions made from them, and food or consumable compositions made from them.

In another aspect, the present invention comprises cocoa ingredients having enhanced levels of resveratrol and/or piceid, for example, chocolate liquor, cocoa powder, or a cocoa extract. The cocoa ingredients can be made from cocoa beans having enhanced levels of resveratrol. The term “cocoa ingredient” refers to any material containing cocoa solids that are derived from cocoa nibs. The term includes chocolate liquor, cocoa powder, cocoa extract, defatted cocoa powder, low fat cocoa powder, alkalized chocolate liquor, alkalized nibs and alkalized cocoa powder. The terms “chocolate liquor” and “cocoa liquor” refer to the viscous substance formed by grinding cocoa nibs. Chocolate liquor is the key ingredient in the manufacture of many chocolate products, for example, milk chocolate, dark chocolate, semi-sweet baking chips, reduced fat chocolate, reduced-sugar or sugar-free chocolate, chocolate-flavored coatings, and baking chocolate.

In a preferred embodiment, a cocoa powder in accordance with the present invention may have a total resveratrol content of about 3 μg/g to about 50 μg/g. A chocolate liquor according to the present invention may have a total resveratrol content of about 2.5 μg/g to about 50 μg/g.

In another aspect, the present invention comprises a cocoa extract having enhanced levels of natural stilbene compounds, especially resveratrol. A cocoa extract having enhanced levels of resveratrol can be made by any method that removes the resveratrol compound from the cocoa solids and fat component thus rendering it in solution. The solution can be further concentrated and the resulting extract used by itself or added to any number of foods, supplements or consumable materials. Concentrations as low as 2× and as high as 25× to about 50× can be produced and used to enhance levels of total resveratrol or any particular stilbene or group of stilbenes. For food products, highly concentrated extracts may optionally employ a bitter-blocker or flavors for masking the typically bitter components that may concentrate with the resveratrol or stilbenes.

The present invention also includes food products containing cocoa ingredients having enhanced levels of resveratrol. The term “food product” includes any edible or consumable product that can be ingested by humans or animals to provide nourishment or provide supplements, and includes but is not limited to chocolate foods, chocolate bars, chocolate candies, steeped cocoa beverages, chocolate drinks, chocolate-flavored foods, chocolate-flavored bars, chocolate-flavored candies, chocolate-flavored drinks, chocolate-coated foods, chocolate-coated bars, chocolate-coated candies, milk chocolate, dark chocolate, baking chocolate, semi-sweet baking chips, baked chocolate products, such as cakes, brownies and breads, reduced-sugar chocolate and reduced-fat chocolate.

In another aspect, the invention includes ingredients or compositions, including pharmaceutical compositions, having natural stilbene compounds derived from Theobroma cacao, which compounds may include trans- and cis-resveratrol, trans- and cis-piceid, trans- and cis-pterostilbene, trans- and cis-piceatannol, or any other stilbene compound or stilbene oligomer occurring naturally in a cacao plant. The invention further includes methods of increasing or stimulating nitric oxide generation and/or nitric oxide synthase protein expression in cells by administering a composition, such as a pharmaceutical composition, or an ingredient having enhanced levels of natural stilbene compounds derived from Theobroma cacao. In another aspect, the invention includes methods of inhibiting the enzymatic activities of COX-1 and/or COX-2 in cells by administering an ingredient or a composition, such as a pharmaceutical composition, having enhanced levels of natural stilbene compounds derived from Theobroma cacao. In a further aspect, the invention includes methods of decreasing NADH/NADPH oxidase activity by administering compositions or ingredients according to the invention. Furthermore, the invention includes methods of blocking LDL oxidation. The methods include administering ingredients or compositions having from about 10 mg to about 200 mg or more of resveratrol per dose in an animal.

The ingredients used in the methods may be prepared by selecting unfermented cocoa beans, or a cocoa bean sample having more than 10% unfermented beans, and processing the beans into a cocoa ingredient having non fat cocoa solids. The cocoa ingredient may be added to a food product. The compositions, including pharmaceutical compositions, may be prepared by selecting unfermented cocoa beans, reducing the beans to a powder, defatting the powder and extracting natural stilbene compounds from the powder. The ingredients and compositions can be prepared from any part of the cacao bean. Compositions made in accordance with the present invention from the cacao shell, for example, can have higher levels of natural stilbene compounds, such as resveratrol and piceid, than compositions prepared in other ways.

### EXAMPLES

### Example 1

### Producing Cocoa Beans Having Enhanced Levels of Total Resveratrol

Cacao beans with enhanced levels of resveratrol can be selected from beans available from growers or can be produced with new treatments as disclosed here. In one example, an unfermented bean available from Mexico is used to enhance resveratrol levels of cocoa compositions. Mexico cacao lavado beans are selected and washed using known methods to remove fermentable sugars. The pulp is removed and the beans are sun-dried. The dried, unfermented beans contain enhanced levels of resveratrol. Mixtures of beans containing more than 10% of cacao lavado beans can also contain enhanced levels of resveratrol.

### Example 2

### Selecting Cocoa Beans Having Enhanced Levels of Total Resveratrol

In another example, cocoa beans having enhanced levels of total resveratrol are selected from unfermented or partially unfermented beans. Specifically, the cocoa beans selected for processing are slaty beans, purple beans, or mixtures of slaty beans and purple beans. Slaty beans are unfermented beans and purple beans are slightly unfermented. The selected beans contain more than 10% of slaty and/or purple beans and are processed into resveratrol rich cocoa ingredients.

### Example 3

### Blending Unfermented and Fermented Cocoa Beans

A cocoa blend having increased levels of total resveratrol can be prepared by selecting a mixture of unfermented and fermented cocoa beans. The blend includes at least 10% unfermented beans, such as Mexico cacao lavado beans having a fermentation index of 0.38, and up to 90% fermented beans, such as Jamaica beans having a fermentation index of 1.44 (from the method noted above). A blend having 10% Mexico cacao lavado beans and 90% Jamaica beans has 4.08 μg/g of total resveratrol, and 0.61 μg/g of trans-resveratrol, or almost 40% more trans-resveratrol than Jamaica beans alone (Table 9). Cocoa bean blends composed of higher levels of unfermented beans have higher levels of total resveratrol. For example, a cocoa blend having 50% unfermented beans has 1.31 μg/g of trans-resveratrol and 6.02 μg/g of total resveratrol. A cocoa blend having 80% unfermented beans has 1.83 μg/g of trans-resveratrol and 7.49 μg/g of total resveratrol, or over 100% more total resveratrol than a sample having no unfermented beans.

Table 9 shows the amounts of trans-resveratrol, trans-piceid and total resveratrol present in cocoa bean blends made up of various mixtures of unfermented and fermented cocoa beans. The “% more” column shows the percentage increase or degree of enhancement in resveratrol content of the cocoa bean blend compared to a cocoa bean sample containing substantially 100% fermented beans.

TABLE 9
Blends of Unfermented and Fermented Cocoa Beans

| % | | % | | | | | | | |
|---|---|---|---|---|---|---|---|---|---|
| Unferm | | Ferm | | trans- | | | | Total | |
| Cocoa | Ferm | Cocoa | Ferm | Resveratrol | | trans-Piceid | | Resveratrol | |
| Beans | Index | Beans | Index | μg/g | % more | μg/g | % more | μg/g | % more |
| 100 | 0.38 | 0 | 1.44 | 2.18 | 395 | 6.28 | 99 | 8.46 | 136 |
| 90 | 0.38 | 10 | 1.44 | 1.97 | 348 | 5.97 | 90 | 7.97 | 122 |
| 80 | 0.38 | 20 | 1.44 | 1.83 | 316 | 5.65 | 79 | 7.49 | 109 |
| 70 | 0.38 | 30 | 1.44 | 1.66 | 277 | 5.34 | 70 | 7.00 | 95 |
| 60 | 0.38 | 40 | 1.44 | 1.48 | 236 | 5.03 | 60 | 6.51 | 81 |
| 50 | 0.38 | 50 | 1.44 | 1.31 | 198 | 4.71 | 50 | 6.02 | 68 |
| 40 | 0.38 | 60 | 1.44 | 1.14 | 159 | 4.40 | 40 | 5.54 | 54 |
| 30 | 0.38 | 70 | 1.44 | 0.96 | 118 | 4.09 | 30 | 5.05 | 41 |
| 20 | 0.38 | 80 | 1.44 | 0.79 | 80 | 3.78 | 20 | 4.56 | 27 |
| 10 | 0.38 | 90 | 1.44 | 0.61 | 39 | 3.46 | 10 | 4.08 | 14 |
| 0 | 0.38 | 100 | 1.44 | 0.44 | 0 | 3.15 | 0 | 3.59 | 0 |

### Example 4

### Chocolate Liquor Having Enhanced Levels of Total Resveratrol

Roasted chocolate liquor is prepared from dried, unfermented Mexico cacao lavado beans. The beans contain 2.18 μg/g trans-resveratrol and 6.28 μg/g trans-piceid. The total resveratrol content of the beans is 8.46 μg/g. The chocolate liquor contains 0.47 μg/g to 0.73 μg/g trans-resveratrol and 5.65 μg/g to 6.02 μg/g trans-piceid, or 71% to 75% of the total resveratrol content of the unroasted, whole beans (see Tables 10 and 10A).

TABLE 10
Resveratrol levels in chocolate liquor and cocoa powder
prepared from unfermented Mexico cacao lavado beans

| | trans- | | Total |
|---|---|---|---|
| | resveratrol | trans-piceid | Resveratrol |
| | (μg/g) | (μg/g) | (μg/g) |
| Medium roasted | 0.73 | 5.65 | 6.38 |
| liquor | | | |
| Cocoa powder* | 1.37 | 10.58 | 11.94 |

*cocoa powder values calculated from chocolate liquor values

The unfermented Mexico cacao lavado beans, while described many years ago, are not used in producing cocoa products except at very low levels in Mexican-sourced cacao bean samples, for example no more than about 10% in most cocoa bean samples. Similarly, other cacao bean-producing regions limit the content of unfermented slaty beans to 10% due to its effect on taste and astringency. Furthermore, International Standards limit the level of slaty and purple unfermented beans to 10% or less (BCCCA, Cocoa Beans: Chocolate Manufacturers Quality Regulations, 1996). Accordingly, products made from cacao bean samples with more than 10% unfermented beans are unusual and are generally considered to be low quality. According to the invention, the use of much higher levels of cacao lavado beans, which are washed, unfermented beans, from more than 10% or any level higher, can lead to cocoa bean compositions with enhanced levels of resveratrol.

The HPLC method previously described by Milbury (Milbury, P E.; Chen, C Y.; Dolnikowski, G G.; Blumberg, J B. Determination of Favonoids and Phenolics and their Distribution in Almonds. J. Agric. Food Chem. 2006, 54: 5027-5033.) was modified to measure t-resveratrol, t-piceid, and piceatannol. All samples were stored in darkness at 4° C. throughout the following operations with some samples ground, both before and after lipid removal with hexane, in order to improve the subsequent extraction. The cocoa powder samples did not require grinding.

For each of the samples, 5.00 g was delipidated with 40 mL hexane for three hours (3 times each). The samples were centrifuged between defatting steps and the hexane decanted. The remaining solid pellet was dried under vacuum at 35° C. to remove any residual hexane. The delipidated pellet was hydrolyzed for 8 hours at 4° C. using 40 mL of a methanol/water/HC; solution (50/46.3/3.7 v/v). The solution was centrifuged and the supernatant collected. The hydrolysis process was repeated and the two resulting supernatants were combined and treated with 3.2 g of sodium bicarbonate to adjust the pH to 5. Organic solvents were removed by rotary evaporation at 35° C. The concentrated hydrolysate (approx. 25 mL) was extracted two times with 125 mL of ethyl acetate. The ethyl acetate layer was evaporated to dryness and redissolved in methanol. Insoluble material was removed by filtration and the eluent solution was used for HPLC analysis.

All HPLC analyses were performed on a Hewlett Packard 1090 instrument equipped with an autosampler, tertiary pump, column heater, and diode array detector. Separation was achieved using a 100×3.0 mm i.d., 3 μm YMC PackPro C-18 RP column, with a solvent gradient of acetonitrile/methanol (50/50 v/v) from 5 to 26% in 0.1% aqueous TFA over 40 min at a flow rate of 0.600 ml/min. The chromatography was monitored at 310 nm. HP hemstation v.10.03 was used for data collection and evaluation. The quantity of trans-resveratrol, trans-piceid, and piceatannol in the cocoa samples was calculated by comparison with absorption data from reference standards (all commercially available) injected at a concentration of 5 μg/mL and a volume of 4 μL. Each sample was analyzed in duplicate.

TABLE 10A
Levels of resveratrol and other stilbenes in chocolate liquor
and cocoa powder prepared from unfermented Mexico
cacao lavado beans and conventional processing

| | Trans- | Trans- | Total | |
|---|---|---|---|---|
| | resveratrol | piceid | resveratrol | Piceatannol |
| Samples | (ug/g) | (ug/g) | (ug/g) | (ug/g) |
| Medium-roast | 0.47 | 6.02 | 6.49 | 4.24 |
| Mexico lavado | | | | |
| liquor | | | | |
| Medium roast | 0.73 | 6.68 | 7.41 | 4.18 |
| Mexico lavado | | | | |
| powder | | | | |

### Example 5

### Cocoa Powder Having Enhanced Levels of Total Resveratrol

The total resveratrol content of a cocoa powder pressed from the chocolate liquor of Example 4 can be calculated. Cocoa liquor comprises an average of about 53% fat and 47% non-fat cocoa solids while press cake cocoa powders typically comprise 12% fat and 88% non fat cocoa powder. The cocoa powder obtained from the above liquor would be expected to contain 1.872 times more resveratrol per gram than the liquor, yielding 11.94 μg total resveratrol per gram of powder. This represents a 40% higher level of total resveratrol than the unprocessed beans. Using a standard serving size of cocoa powder of 10 grams, this powder would contain 119 μg of total resveratrol per serving. This level of resveratrol is about 6.45 times higher than the average of U.S. cocoa powders (see Tables 6 and 7). Clearly, the use of the unfermented beans can have significant impact on enhancing resveratrol levels. As shown in Table 10A, cocoa powder produced from cocoa lavado chocolate liquor has 7.4 μg/g total resveratrol or more. Using a standard serving size of log yields about 74 to about 119 μg/g total resveratrol. This level is 4× to about 6.45× the levels we find in natural cocoa powder.

### Example 6

### Treatments to Enhance Resveratrol During Cacao Bean Production, Harvesting or Processing

The levels of resveratrol and/or piceid in cacao beans can be manipulated or enhanced by a number of means. Exemplary treatments or conditions for any cacao bean and in particular for unfermented cacao beans include: mold-infestation; UV irradiation; aluminum chloride treatment; aluminum sulfate treatment; ozone treatment; salicylic acid treatment; refrigerated storage; heat treatment; and any combination of these.

In one example, freshly harvested cacao beans are removed from pods, washed, and irradiating under UV-C or 254 nm wavelength light (for example, low pressure mercury 6 W lamp, or germicidal lamp) for about 1 minute, or about 1 to about 15 minutes, with optional turning to expose all sides of the bean to UV light. The UV-induction or enhancement of resveratrol levels can be examined by quantitative HPLC using common reverse phase C-18 columns and a water/acetonitrile gradient from 95/5 to 30/70.

Similarly, beans collected from pest or fungus infested pods or samples may contain higher levels of resveratrol from the disease-resistance function of the stilbenes. Many fungal infestations are known to affect Theobroma cacao plants, including Moniliophthora roreri (“Frosty Pod”), Crinipellis perniciosa (“Witches' Broom”), Ceratocystis fimbriata (“Mal de machete” or “Ceratocystic wilt”), and Verticillium dahliae. A preferred microbe or inoculant for this treatment is a Botrytis inoculant. Bean samples taken from infested plants or pods or beans intentionally infested with one or more of such fungus can be tested for levels of resveratrol compared to untreated or normal beans or plants.

### Example 7

### Production of Cocoa Ingredients with Elevated Resveratrol in Material that has been Alkali Treated

The chocolate liquor, cocoa nibs, and cocoa powder prepared by methods of the invention can be alkalized. For example, the cocoa powder of the previous examples can be alkalized using sodium carbonate, potassium carbonate, or any other alkali solution or suspension known in the art. The cocoa powder is “lightly” alkalized, or treated with an alkali solution to raise the pH to pH 7.0. The lightly alkalized cocoa ingredient contains 1.0 μg/g trans-resveratrol, 5.1 μg/g trans-piceid and 6.2 μg/g total resveratrol, or only about ¼ less total resveratrol than a non-alkalized ingredient (Table 11). Increasing the level of alkalization, for example to a pH of 7.4, or further to a pH of 7.9, results in a substantial decrease in the amount of total resveratrol present in the ingredient. A “medium”-alkalized ingredient, or an ingredient alkalized to a pH of about 7.4, contains only about 50% as much total resveratrol as a non-alkalized ingredient, and a “heavily” alkalized ingredient contains only 35% as much total resveratrol. Thus, light alkalization can improve the taste and/or flavor of the cocoa ingredients of the invention without forfeiting the benefits of the enhanced stilbene or resveratrol content. The alkalization conditions and other conditions, such as oxygen concentration, time, and alkalizing agent, can be manipulated to produce a cocoa ingredient benefiting from the effects of alkalization while maintaining enhanced levels of resveratrol.

Table 11 shows the amount of trans-piceid, trans-resveratrol and total resveratrol present in a cocoa ingredient subjected to light, medium and heavy alkalization, compared to an ingredient that is not alkalized. The “% Remaining” column identifies the percentage of resveratrol remaining in the ingredient after various levels of alkalization compared to a non-alkalized ingredient.

TABLE 11
Impact of Alkalization on Resveratrol Content

| | | | | trans- | | Total | |
|---|---|---|---|---|---|---|---|
| Level of | | trans-Piceid | | Resveratrol | | Resveratrol | |
| Alkali- | | | % | | % | | % |
| zation | pH | μg/g | Remaining | μg/g | Remaining | μg/g | Remaining |
| None | 5.6 | 6.6 | 100 | 1.9 | 100 | 8.5 | 100 |
| Light | 7.0 | 5.1 | 77 | 1.0 | 53 | 6.2 | 73 |
| Medium | 7.4 | 3.7 | 56 | 0.6 | 32 | 4.4 | 52 |
| Heavy | 7.9 | 2.8 | 42 | 0.2 | 10 | 3.0 | 35 |

### Example 8

### Chemical Extraction of Materials with Elevated Resveratrol for the Purposes of Obtaining an Extract

The ingredients of the invention having enhanced levels of resveratrol can be further processed to produce a cocoa extract having enhanced resveratrol levels. Extraction can be performed using an alcohol, water, hot water, ethanol, methanol, ethyl acetate, isopropanol, isopropyl alcohol, and any other aqueous or organic solvent used in the food processing industry, or a combination thereof. Chocolate liquor made from cacao beans containing more than 10% unfermented beans is reduced to cocoa powder, and the cocoa powder is defatted. The defatted powder is dried and then intermixed with desired solvent with mechanical stirring or another appropriate technique. The resulting liquid and solid portions are separated by decantation to produce a liquid extract. The resulting extract contains enhanced levels of resveratrol (see Table 12). Cocoa extracts prepared in accordance with the invention may contain at least about 10 μg/g of total resveratrol, or at least about 20 μg/g, or 30 μg/g, or 40 μg/g, or 50 μg/g, or 60 μg/g or more of total resveratrol. The compositions of the invention also include cocoa extracts having at least about 10 μg/ml of total resveratrol, or at least about 20 μg/ml, or 30 μg/ml, or 40 μg/ml, or 50 μg/mil, or 60 μg/ml or more of total resveratrol The cocoa extract may be added to a material without substantially affecting or degrading the flavor, mouthfeel, or the physical or taste attributes of the material. For example, the cocoa extract may be added to a beverage, such as a clear beverage, without changing the flavor or the visual appearance of the beverage. Alternatively, the cocoa extract may be used as a flavoring agent and dispensed from a liquid dispenser such as a hand pump.

TABLE 12
Predicted Solvent Extraction on Resveratrol
Content (with a 10-fold concentration)

| | | | Total |
|---|---|---|---|
| | trans- | trans-Piceid | Resveratrol |
| | Resveratrol(μg/g) | (μg/g) | (μg/g) |
| Cocoa | 1.37 | 10.58 | 11.94 |
| Powder | | | |
| Solvent | 13.7 | 105.8 | 119.4 |
| Extract | | | |
| 10X | | | |

Accordingly, especially in combination with other methods described here, the optional solvent extraction of cocoa powders made from cacao bean samples containing 10% or more of unfermented beans, such as Mexican cacao lavado beans, results in food or ingredient extracts with advantageously high levels of resveratrol. The use of desolventizing methods, conditions, and/or compositions or solvents can also be used, for example, to improve the final characteristics or properties of the extract or a product containing it.

### Example 8a

### Ethanol Extraction of Unfermented Lavado Cocoa Nibs to Produce Extracts with Enhanced Stilbene Content

One hundred grams (100 g) of unroasted shell-free cocoa nibs (Sample #1) from unfermented Mexican lavado beans were solvent extracted with 200 ml of food grade ethanol. The nibs were soaked for 24 hours followed by mixing for six hours at room temperature (25° C.). The nibs and solvent mixture was gravity filtered using Whatman #4 filter paper to yield a particulate free ethanol extract solution (Sample #2). The ethanol extract was evaporated under vacuum to a volume of 12 ml to yield a 16.6× concentrate (Sample #3). Ten ml (10 ml) of the concentrate was diluted with 190 ml of water and freeze-dried to dryness yielding a powder extract (Sample #4). The samples were analyzed for trans-resveratrol, trans-piceid, and piceatannol and the results are shown in Table 12a. This extraction procedure yields roughly a 10-fold concentration of stilbene content based on the stilbene amount in the starting nibs. This extraction procedure is not optimized and it would be reasonable to predict extracts with 5-fold more stilbene content in the final extracts. The ethanol concentrates and/or powder extracts can be used as ingredients to produce food products with enhanced stilbene content. Alternatively, one could employ one or more available techniques for isolating or purifying stilbenes or resveratrol compounds, such as HPLC separation, solvent extraction, countercurrent chromatography, and/or supercritical fluid extraction (SFE).

TABLE 12A
Cocoa extract of Mexican cocoa lavado
with enhanced stilbene content.

| | Trans- | | Trans- | | Total | | | |
|---|---|---|---|---|---|---|---|---|
| Sample | resveratrol | | piceid | | resveratrol | | Piceatannol | |
| #1 Lavado | 1.0 | ug/g | 2.9 | ug/g | 3.9 | ug/g | 1.9 | ug/g |
| Nibs | | | | | | | | |
| #2 Ethanol | 0.04 | ug/ml | 0.07 | ug/ml | 0.11 | ug/ml | 0.08 | ug/ml |
| extract | | | | | | | | |
| #3 16.7X | 1.1 | ug/ml | 1.6 | ug/ml | 2.7 | ug/ml | 1.6 | ug/ml |
| concentrate | | | | | | | | |
| #4 Powder | 16.7 | ug/g | 30.9 | ug/g | 47.6 | ug/g | 19.2 | ug/g |
| Extract | | | | | | | | |

### Example 9

### Products Made from Ingredients or Extracts Enriched in Cacao Resveratrol

Any of the cocoa ingredients produced by the methods of the invention can be incorporated into a food product, such as a chocolate food product, a chocolate bar, a chocolate-flavored product, a chocolate confectionery, a chocolate-flavored confectionery, a beverage, a chocolate beverage, a chocolate-flavored beverage, a dietary supplement, a chocolate-coated product, a baked chocolate product, such as a cake, brownie or bread, a low fat chocolate product or a low-sugar chocolate product. The resulting food products contain higher levels of resveratrol than a product made from conventional cocoa ingredients.

In one example, an ingredient is prepared from a bean sample containing substantially all Mexico cacao lavado beans. The ingredient may be cocoa powder, chocolate liquor, cocoa mass, or a cocoa extract, but many other cocoa ingredients could also be produced and used. One way of using the ingredient is in supplementing a food product, such as a chocolate food product. For example, a cocoa powder made from substantially all Mexico cacao lavado beans may be used to supplement a chocolate food product in order to improve the health properties of or increase the levels of beneficial cocoa compounds in a final food product. For example, a milk chocolate bar can be supplemented with about 5% of the cocoa powder made from Mexican cacao lavado beans. The taste and mouthfeel properties can be the same as a conventional milk chocolate bar, but the levels of resveratrol and cocoa polyphenols can be increased substantially. Other food compositions can similarly be supplemented, and the exemplary 5% amount noted here can change, for example, 10% to 1% by weight of a cocoa powder from cacao lavado or unfermented beans can be used, or 7% to 5%, or 5% to 3%, or 3% to 1%, or about 2%, or about 1%. The resulting food product contains enhanced levels of resveratrol compared to conventional products of the same type.

### Example 10

### Baked Products Made from Cocoa Ingredients Enriched with Cocoa Resveratrol

Any of the cocoa ingredients produced by the methods of the invention can be incorporated into a baked food product such as a cake, brownie, bread, or other food product or ingredient subjected to a baking process. The resulting food products will contain higher levels of resveratrol and other stilbenes than a product made from conventional ingredients.

From the data above, one would logically predict that heating compositions containing stilbenes would reduce the levels of stilbenes and resveratrol present. However, surprisingly, stilbene contents are not totally destroyed during the baking process. In one example, chocolate cakes are prepared from conventional natural cocoa powder using two types of leavening agents; baking soda and baking powder (see Table 13) and other typical ingredients such as: flour, sugar, water, margarine, eggs, salt, and vanilla. The cake batters containing the cocoa powder are baked for 35 minutes at 350° F. The resulting cakes retain >88% of the resveratrol present in the original coco powder used in the recipe. In addition, >50% of the piceid and piceatennol remain after the baking process. There may be an advantageous retention of stilbene content during baking by using baking soda instead of baking powder but in either case significant amounts of stilbenes are retained during baking.

TABLE 13
Baking compositions and Baking Process Effects

| | | t- | | t- | | | |
|---|---|---|---|---|---|---|---|
| | | resveratrol | | piceid | | Piceatennol | |
| Sample Description | pH | (ppm) | % | (ppm) | % | (ppm) | % |
| Cake batter - baking | 7.5 | 0.08 | 100 | 0.21 | 100 | 0.19 | 100 |
| soda | | | | | | | |
| Baked Cake - baking | 8.8 | 0.09 | 112 | 0.24 | 114 | 0.11 | 58 |
| soda | | | | | | | |
| Cake batter - baking | 6.3 | 0.09 | 100 | 0.22 | 100 | 0.09 | 100 |
| powder | | | | | | | |
| Baked Cake - baking | 6.4 | 0.08 | 89 | 0.13 | 59 | 0.05 | 56 |
| powder | | | | | | | |

% refers to the percent of the compound retained compared to initial

In another example, a chocolate cake can be prepared with cocoa ingredients with enriched resveratrol content resulting in a baked product with higher levels of resveratrol than a cake baked with conventional cocoa ingredients. In this case, the cocoa ingredients are made from largely unfermented cocoa beans. These ingredients can be, but limited to: cocoa powder, cocoa extract, or chocolate chips. The degree of resveratrol enrichment will be proportional to the percentage of unfermented beans used to prepare the cocoa ingredient as illustrated in Table 9.

The examples presented above and the contents of the application define and describe examples of the many cocoa compositions, products, and methods that can be produced or used according to the invention. None of the examples and no part of the description should be taken as a limitation on the scope of the invention as a whole or of the meaning of the following claims.

What is claimed is:

1. A method of enhancing the level of resveratrol present in a food product, comprising selecting cacao beans having about 1 μg/g or more of trans-resveratrol and/or about 5 μg/g or more of total resveratrol, and supplementing or adding non fat cocoa solids produced from these beans to a food product.

2. The method of claim 1, further comprising making a chocolate liquor, a cocoa powder or a cocoa extract from the selected cacao beans.

3. The method of claim 2, wherein the chocolate liquor has about 6.4 μg/g or more of total resveratrol.

4. The method of claim 2, wherein the chocolate liquor has about 0.4 μg/g or more of trans-resveratrol.

5. The method of claim 2, wherein the cocoa powder has about 7 μg/g or more of total resveratrol.

6. The method of claim 2, wherein the cocoa powder has about 0.7 μg/g or more of trans-resveratrol.

7. The method of claim 1, wherein the cacao beans comprise at least 10% unfermented beans.

8. The method of claim 1, wherein the cacao beans comprise at least 10% slaty beans.

9. The method of claim 1, wherein the cacao beans comprise at least 10% slaty beans and purple beans.

10. The method of claim 1, wherein the beans are used to produce a cocoa ingredient selected from chocolate liquor, cocoa powder, cocoa extract, low fat cocoa powder, defatted cocoa powder, and non fat cocoa solids.

11. The method of claim 2, wherein the cocoa powder is treated with alkaline conditions.

12. The method of claim 2, wherein the chocolate liquor is treated with alkaline conditions.

13. The method of claim 2, wherein the cocoa beans or nibs are treated with alkaline conditions.

14. A food product made from the method of any one of claims 3 or 4.

15. The food product of claim 14, wherein the food product is a chocolate product, a chocolate-flavored product, a chocolate confectionery, a chocolate-flavored confectionery, a beverage, a chocolate beverage, a chocolate-flavored beverage, a dietary supplement, a chocolate-coated product, a baked food product, a chocolate cake or brownie, a low fat chocolate product, or a low-sugar chocolate product.

16. A cocoa ingredient having enhanced levels of natural stilbene compounds.

17. The cocoa ingredient of claim 16 having enhanced levels of total resveratrol and/or trans-resveratrol.

18. The cocoa ingredient of claim 17, wherein the ingredient comprises a chocolate liquor, a cocoa powder, a low fat cocoa powder, a defatted cocoa powder, or a cocoa extract.

19. The cocoa ingredient of claim 18, wherein the cocoa ingredient is cocoa powder, and wherein the cocoa powder has about 7 μg or more of total resveratrol per gram of cocoa powder.

20. The cocoa ingredient of claim 18, wherein the cocoa ingredient is cocoa powder, and wherein the cocoa powder has about 0.7 μg or more of trans-resveratrol per gram of cocoa powder.

21. A food product comprising the cocoa ingredient of either one of claims 19 or 20.

22. The food product of claim 21, wherein the food product is a chocolate product, a chocolate-flavored product, a chocolate confectionery, a chocolate-flavored confectionery, a beverage, a chocolate beverage, a chocolate-flavored beverage, a dietary supplement, a chocolate-coated product, a baked food product, a chocolate cake or brownie, a low fat chocolate product or a low-sugar chocolate product.

23. The cocoa ingredient of claim 18, wherein the cocoa ingredient is chocolate liquor, and wherein the chocolate liquor has at least about 6.4 μg or more of total resveratrol per gram of liquor.

24. The cocoa ingredient of claim 18, wherein the cocoa ingredient is chocolate liquor, and wherein the chocolate liquor has about 0.4 μg or more of trans-resveratrol per gram of liquor.

25. A food product comprising the cocoa ingredient of either one of claims 23 or 24.

26. The food product of claim 25, wherein the food product is a chocolate product, a chocolate-flavored product, a chocolate confectionery, a chocolate-flavored confectionery, a beverage, a chocolate beverage, a chocolate-flavored beverage, a dietary supplement, a chocolate-coated product, a baked food product, a chocolate cake or brownie, a low fat chocolate product or a low-sugar chocolate product.

27. The cocoa ingredient of claim 18, wherein the cocoa ingredient is a cocoa extract, and wherein the cocoa extract has at least about 4 μg or more of total resveratrol per gram of extract.

28. The cocoa ingredient of claim 18, wherein the cocoa ingredient is a cocoa extract, and wherein the cocoa extract has about 30 μg or more of total resveratrol per gram of cocoa extract.

29. The cocoa ingredient of claim 18, wherein the cocoa ingredient is a cocoa extract, and wherein the cocoa extract has about 50 μg or more of total resveratrol per gram of cocoa extract.

30. The cocoa ingredient of claim 18, wherein the cocoa ingredient is a cocoa extract, and wherein the cocoa extract has about 10 μg or more of trans-resveratrol per gram of cocoa extract.

31. The cocoa ingredient of claim 16 having enhanced levels of trans-piceid.

32. The cocoa ingredient of claim 16 having enhanced levels of pterostilbene.

33. The cocoa ingredient of claim 16 having enhanced levels of piceatannol.

34. A method of enhancing the level of resveratrol in a cocoa composition, comprising preparing a cacao bean sample having more than 10% of washed, unfermented cacao beans, and processing the sample into a cocoa composition having non fat cocoa solids in an edible form.

35. A method of enhancing the levels of resveratrol in products made from cacao beans, comprising selecting unfermented cacao beans, irradiating the beans with UV light, and preparing a cocoa composition from the beans.

36. The method of claim 35, wherein the cocoa composition is a chocolate liquor, a cocoa powder, or an extract from the beans.

37. The method of claim 36, wherein UV-B light is used.

38. The method of claim 36, wherein UV-C light is used.

39. The method of claim 36, wherein UV light is used for between about 1 minute to about 15 minutes.

40. A method of enhancing the levels of resveratrol in products made from cacao beans, comprising selecting unfermented cacao beans and treating them with one or more of: inoculating with a fungus or microbe; storage for 1 or more days at about 0° C.; treatment with aluminum chloride; treatment with aluminum sulfate, and producing a food ingredient or extract from the treated cacao beans.

41. The method of claim 40, wherein a Botrytis inoculate is used to treat the beans.

42. The method of claim 40, wherein the selected treatment is combined with a treatment with UV light.

43. A method of enhancing nitric oxide generation in cells comprising administering an ingredient having enhanced levels of resveratrol derived from Theobroma cacao, wherein said ingredient is prepared by a method comprising:
selecting a bean sample having more than 10% unfermented cocoa beans; and
processing the bean sample into a cocoa composition having non fat cocoa solids in an edible form.

44. A method of enhancing nitric oxide generation in cells comprising administering a pharmaceutical composition comprising an organic extract having enhanced levels of natural stilbene compounds from Theobroma cacao, wherein the extract is prepared by:
selecting a bean sample having more than 10% unfermented cocoa beans;
reducing the bean sample to a powder;
defatting the powder; and
extracting natural stilbene compounds from the powder.

45. A method of inhibiting the enzymatic activity of COX-1 or COX-2 in cells comprising administering an ingredient having enhanced levels of resveratrol derived from Theobroma cacao, wherein said ingredient is prepared by a method comprising:
selecting a bean sample having more than 10% unfermented cocoa beans; and
processing the bean sample into a cocoa composition having non fat cocoa solids in an edible form.

46. A method of decreasing NADH/NADPH oxidase activity in cells comprising administering an ingredient having enhanced levels of resveratrol derived from Theobroma cacao, wherein said ingredient is prepared by a method comprising:
selecting a bean sample having more than 10% unfermented cocoa beans; and
processing the bean sample into a cocoa composition having non fat cocoa solids in an edible form.

47. A pharmaceutical composition comprising resveratrol, the resveratrol prepared by:
selecting a bean sample having more than 10% unfermented cocoa beans; and
extracting resveratrol compounds from the bean sample.

48. A method of supplementing the levels of beneficial resveratrol in a food product comprising preparing a cocoa ingredient from a sample of cacao beans containing about 90% or more unfermented cacao beans, and adding about 10% to about 3% by weight of the cocoa ingredient to a food product during the processing into a final product.

49. The method of claim 48, wherein the cocoa ingredient is a cocoa powder.

50. The method of claim 48, wherein the food product is a chocolate product.

51. The method of claim 49, wherein about 5% to about 3% by weight of the cocoa ingredient is added.

52. The method of claim 48, wherein preparing the cocoa ingredient further comprises a solvent extraction to increase the resveratrol levels.

53. A method of enhancing levels of total resveratrol in a food product, comprising:
selecting a bean sample having more than 10% unfermented cocoa beans;
reducing the bean sample to a powder;
solvent extracting the powder to produce an extract having enhanced levels of resveratrol; and
supplementing or adding the extract to a food product.

54. The method of claim 52, wherein the extract has about 10 μg or more of total resveratrol per gram of extract.

55. The method of claim 52, wherein the extract has about 60 μg or more of total resveratrol per gram of extract.

56. The method of claim 52, wherein the solvent comprises an alcoholic solvent, an aqueous solvent, or both an alcoholic and aqueous solvent.

57. A method of enhancing levels of natural stilbene compounds in a food product, comprising:
selecting a bean sample having more than 10% unfermented cocoa beans;
reducing the bean sample to a powder;
solvent extracting the powder to produce an extract having enhanced levels of resveratrol; and
supplementing or adding the extract to a food product.

58. The method of claim 57, wherein the extract has about 3 μg or more of natural stilbene compounds per gram of extract.

59. The method of claim 57, wherein the extract has about 50 μg or more of natural stilbene compounds per gram of extract.

60. The method of claim 57, wherein the solvent comprises an alcoholic solvent, an aqueous solvent, or both an alcoholic and aqueous solvent.

61. The cocoa ingredient of claim 16 having levels of trans-piceid at 6 μg/g or more.

62. The cocoa ingredient of claim 16 having levels of piceatannol at 4 μg/g or more.

63. The cocoa ingredient of claim 16 that is a cocoa powder having piceatannol at 4 μg/g or more.

64. The cocoa ingredient of claim 16 that is a chocolate liquor having piceatannal at 4 μg/g or more.

65. A method of producing a baked chocolate product with enhanced levels of stilbene compounds compared to a conventional baked chocolate product, comprising mixing a cocoa ingredient having an enhanced level of stilbenes with a batter at about pH 7.5 or higher, and baking the batter at about 300 F or higher to form a cake.

66. The method of claim 65, wherein the batter comprises flour, sugar, water, margarine, eggs, salt, and vanilla.

67. The method of claim 65, wherein baking soda is used as a leavening agent to adjust the pH level.

68. A method of producing a baked chocolate product with enhanced levels of stilbene compounds compared to a conventional baked chocolate product, comprising mixing a cocoa ingredient having an enhanced level of stilbenes with a batter at about pH 6.4 or higher, and baking the batter at about 300 F or higher to form a cake.

69. The method of claim 68, wherein the batter comprises flour, sugar, water, margarine, eggs, salt, and vanilla.

70. The method of claim 68, wherein baking powder is used as a leavening agent to adjust the pH level.
